(19) United States
(12) Patent Application Publication
(10) Pub. No.: US 2005/0228399 A1
(43) Pub. Date: Oct. 13, 2005

(54) Guiding device for use in anterior cruciate knee ligament reconstruction

(76) Inventors: Toshikazu Kubo, Kyoto-shi (JP); Kunio Hara, Kyoto-shi (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/061,598

(22) Filed: Feb. 22, 2005

(30) Foreign Application Priority Data
Apr. 12, 2004 (JP) 2004-116659

(51) Int. Cl. A61B 17/58

(52) U.S. Cl. 606/96

(57) ABSTRACT

A guide pin guiding device, for guiding a guide pin when a bone hole for a PLB is prepared in the femur, includes an insertion portion, for being inserted into a bone hole for the AMB prepared in the femur, at one end of an elongate main body thereof. The elongate main body is formed with a guide pin insertion hole for the guide pin to be inserted therein to prepare a bone hole for the PLB in the femur. The guide pin insertion hole is open to the outside of the elongate main body. The guide pin insertion hole includes a guide pin guiding portion and a slot portion extending from the guide pin guiding portion to the side edge of the elongate main body. In addition, a reamer guiding device for guiding a reamer when preparing a bone hole for the PLB in the femur can be used with the guide pin guiding device.

### BACKGROUND OF THE INVENTION

1. Field of the Invention

The present invention relates to a guiding device for guiding a guide pin when a bone hole, for a posterolateral fiber bundle, is prepared in the femur during an anterior cruciate knee ligament reconstruction procedure wherein the anterior cruciate knee ligament is reconstructed by being divided into an anteromedial fiber bundle and a posterolateral fiber bundle.

2. Description of the Related Art

The anterior cruciate ligament (ACL) of the knee is located at the internal front surface of the knee joint and connects the femur to the tibia thereby to restrict a range of flexural movement in the knee joint. The ACL is likely to be injured by hard exercise such as sports, etc. and may sometimes result in rupture. In the anterior cruciate knee ligament reconstructive surgery carried out when the ACL is ruptured, a tendon which can serve as a substitute for the ACL is taken from an appropriate portion of the patient body and the taken tendon is grafted in the knee joint. In the conventional anterior cruciate knee ligament reconstruction procedure, only one tendon has been taken from the appropriate portion of the patient body and joined at one end thereof to the bone hole in the femur and at the other end to the bone hole in the tibia (for example, see Japanese Unexamined Patent Publication No. 2001-25478 and Japanese Unexamined Patent Publication No. 5-184521).

In the conventional procedure, although one tendon was grafted in the knee joint, it has been found that only one grafted tendon cannot satisfactorily function as the ACL. That is, the ACL consists of two bundles, an anteromedial fiber bundle (AMB) and a posterolateral fiber bundle (PLB), which mutually share functions in accordance with the angle of the knee joint, and only one grafted tendon is unable to completely substitute for the ACL to achieve the functions. Therefore, there has been proposed a constructive procedure to reconstruct the AMB and the PLB separately in the reconstruction of the ACL.

In the conventional ACL reconstruction procedure wherein the AMB and the PLB are reconstructed separately, both the bone hole for the AMB and the bone hole for the PLB in the femur has been prepared from the anterior side of the knee joint fixed in the bent position. As the bone hole for the AMB is located at the isometric point of the intercondylar fossa, it is easy to prepare a bone hole at an appropriate site from the anterior side. However, as the bone hole for the PLB is preferably located in the vicinity of femoral posterior condylar joint cartilage edge due to the need of the original functions to bear a strong tension applied thereto in the extended position of the knee, it is difficult to drill the hole in the bone from the anterior side. Therefore, it was likely to overlap the bone hole for the AMB and the possibility of damaging the posterior cortex of lateral femoral condyle existed.

The inventors of the present invention considered the problems in preparing the bone hole for the PLB in the femur by an anterior approach as described above. As a result, they found that a satisfactory result can be obtained by drilling a hole with a technique which could be called a “posteromedial approach” wherein the hole is drilled from the inside of the knee joint toward the vicinity of the femoral posterior condylar joint cartilage edge. However, considerable skill was required for medical doctors to determine a proper position in the vicinity of the femoral posterior condylar joint cartilage edge, which serves as a target point.

### SUMMARY OF THE INVENTION

Therefore, an object of the present invention is to enable the formation of bone holes for the PLB in the femur during the posteromedial approach without requiring great skill.

According to a first aspect of the present invention, there is provided a guide pin guiding device, for guiding a guide pin when a bone hole for a posterolateral fiber bundle is prepared in the femur during a reconstruction procedure in which an anterior cruciate knee ligament is reconstructed by being divided into an anteromedial fiber bundle and a posterolateral fiber bundle, which includes an elongate main body; an insertion portion formed at an end of the elongate main body to be inserted into a bone hole prepared in the femur for the anteromedial fiber bundle; and a guide pin insertion hole formed in a portion of the elongate main body adjacent to the insertion portion for the guide pin to be inserted therein to prepare a bone hole for the posterolateral fiber bundle in the femur and being open to the outside of the elongate main body.

In use of the above-mentioned guide pin guiding device, a bone, hole for a grafted tendon fixed therein, is prepared in the tibia from an anterior side by a normal surgical procedure with the knee joint of a patient bent at a flexion angle of 90°, and the bone hole of the tibia is shared by the AMB and the PLB. That is, a guide pin (K-wire) is pierced into the tibia towards a predetermined target point, and a hole pierced by the guide pin is then enlarged to a predetermined diameter by a reamer to prepare a bone hole, which is shared by the AMB and the PLB. On the other hand, separate bone holes are prepared in the femur for the AMB and the PLB. A position where the bone hole for the AMB is prepared in the femur is an isometric point in an intercondylar fossa and, by way of a surgical procedure similar to one used for preparing the bone hole in the femur, a guide pin (K-wire) is pierced in the femur from an anterior side of the knee joint and the hole pierced by the guide pin is enlarged to a predetermined diameter by a reamer to prepare a bone hole for the AMB. With reference to the position of the bone hole for the AMB, a bone hole for the PLB is then prepared in the femur. That is, the guide pin guiding device according to the present invention is used to pierce the guide pin into the femur from the posteromedial side of the knee joint and prepare the bone hole having a predetermined diameter by means of the reamer with the aid of the guide pin. More specifically, the insertion portion at the forward end of the guide pin guiding device is inserted from the anterior side into the bone hole for the AMB already prepared in the femur. The guide pin guiding device is formed with the guide pin insertion hole for the guide pin for preparing the bone hole for the PLB. The guide pin insertion hole of the guide pin guiding device is formed to orient the guide pin inserted therein toward the vicinity of the femoral posterior condylar joint cartilage edge when the guide pin guiding device is properly mounted. The direction of piercing the guide pin into the guide pin guide hole of the guide pin guiding device in this case is somewhat oriented inwards from the back of the knee joint. The use of the guide pin guiding device enables even medical doctors who have comparatively little experience to easily and properly prepare the bone hole for the PLB in the femur by way of the posteromedial approach.

In the above-mentioned guide pin guiding device, the guide pin insertion hole preferably includes a guide pin guiding portion and a slot portion extending from the guide pin guiding portion to a side edge of the elongate main body.

Provision of a slot portion makes it possible to remove the guide pin guiding device when the guide pin is inserted into the guide pin insertion hole.

In the above-mentioned guide pin guiding device, a plurality of the guide pin insertion holes may be formed in the elongate main body.

Provision of a plurality of guide pin insertion holes makes it possible to select an appropriate one of the guide pin insertion holes depending on a body shape or other feature of the patient, thereby to more easily determine an optimal position of the bone hole for the PLB in the femur to the patient.

According to a second aspect of the present invention, there is provided a reamer guiding device for guiding a reamer for preparing a bone hole for a posterolateral fiber bundle in a femur during a reconstruction procedure in which an anterior cruciate knee ligament is reconstructed by being divided into an anteromedial fiber bundle and the posterolateral fiber bundle, which includes an outer tubular body formed with a through-hole extending therethrough from one end to the other end thereof for a guide pin and an auxiliary inserting tool to be inserted therein, the guide pin pierced in the femur to prepare a bone hole for the posterolateral fiber bundle therein; and a handle portion provided at one end of the outer tubular body.

By means of the above-mentioned reamer guiding device, the bone hole for the PLB can be prepared in the femur without possibility of damaging to the articular capsule tissue, by inserting the main body of the reamer guiding device over the guide pin which is guided and pierced into the femur by means of the guide pin guiding device, inserting the reamer into the main body of the reamer guiding device, and reaming the femur while holding the handle portion of the reamer guiding device.

According to a third aspect of the present invention, there is provided a combination of the above-mentioned guide pin guiding device and the above-mentioned reamer guiding device.

If the guide pin guiding device and the reamer guiding device are combined, it is possible to easily and assuredly carry out the operations of determining a position of a bone hole for the PLB in the femur and actually preparing the bone hole in the femur by the posteromedial approach.

### BRIEF DESCRIPTION OF THE DRAWINGS

The above and other objects, features and advantage of the present invention will be described in more detail below based on the preferred embodiments of the present invention with reference to the accompanying drawings, wherein:

FIG. 1 is a side view of a guiding device for a guide pin according to the present invention;

FIGS. 2A and 2B are plan views showing the guide pin guiding devices for the left femur and for the right femur, respectively;

FIGS. 3A and 3B are cross-sectional views showing the guide pin guiding devices for the left femur (taken along a line III-III of FIG. 2A as viewed from a direction indicated by an arrow) and for the right femur, respectively;

FIG. 4 is a cross-sectional view taken along a line IV-IV of FIG. 1 as viewed from a direction indicated by an arrow;

FIG. 5 is a side view of a guiding device for a reamer according to the present invention;

FIGS. 6A and 6B are plan views showing the reamer guiding devices for the left femur and for the right femur, respectively;

FIG. 7 is a top view of an inner tube used in combination with the reamer guiding device of FIG. 5;

FIG. 8 is a cross-sectional view taken along a line VIII-VIII of FIG. 7 as viewed from a direction indicated by an arrow;

FIG. 9 is a fragmentary perspective view of the cap side end of the inner tube of FIG. 7;

FIG. 10 is a cross-sectional view taken along a line X-X of FIG. 5 as viewed from a direction indicated by an arrow;

FIG. 11 is a perspective view for illustrating a first step (of introducing the guide pin guiding device) for preparing a bone hole for the PLB in the femur using the guide pin guiding device according to the present invention;

FIG. 12 is a perspective view for illustrating a subsequent step (of piercing the guide pin) after the step illustrated in FIG. 11;

FIG. 13 is a perspective view for illustrating a subsequent step (of inserting the inner tube) after the step illustrated in FIG. 12;

FIG. 14 is a perspective view for illustrating a subsequent step (of introducing the reamer guiding device) after the step illustrated in FIG. 13;

FIG. 15 is a perspective view for illustrating a subsequent step (of introducing the reamer) after the step illustrated in FIG. 14; and

FIG. 16 is a perspective view for illustrating a state in which the preparation of the bone hole for the PLB in the femur is completed.

### DETAILED DESCRIPTION OF THE PREFERRED EMBODIMENTS

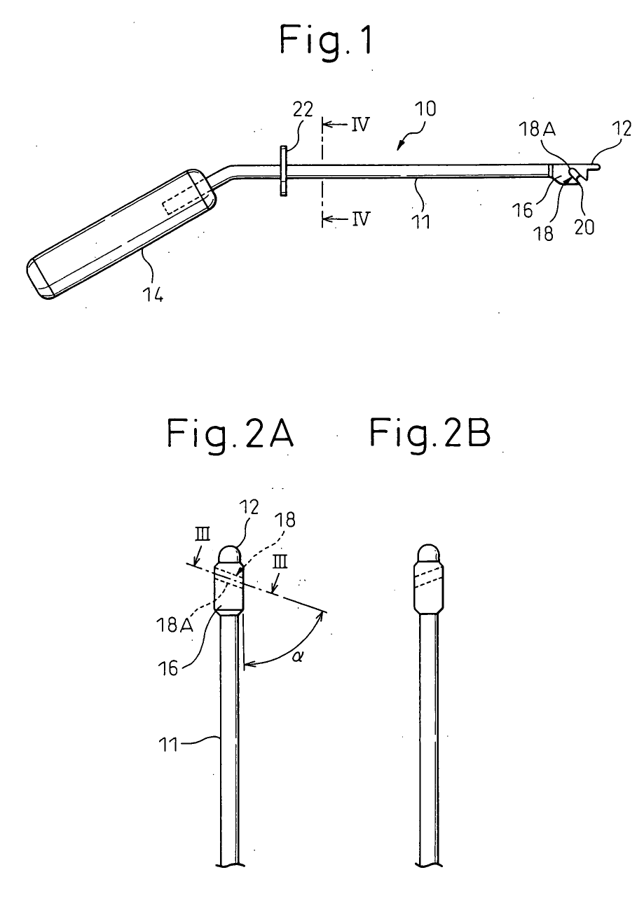

FIG. 1 shows a guide pin guiding device for guiding a guide pin when a bone hole for a posterolateral fiber bundle (PLB) is prepared in the femur during a procedure in which an anterior cruciate knee ligament (ACL) is reconstructed by being divided into an anteromedial fiber bundle (AMB) and a posterolateral fiber bundle (PLB).

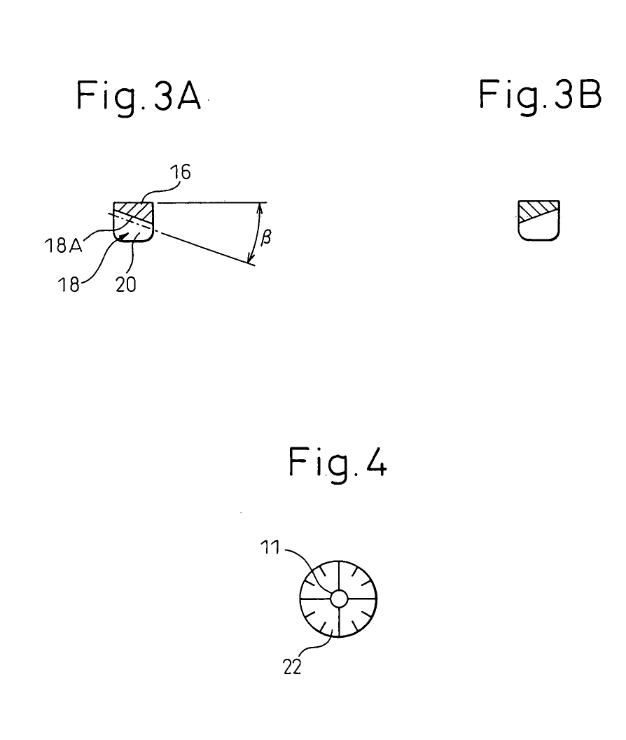

The guide pin guiding device 10 includes an elongated main body 11. The main body 11 is formed at one end thereof with a protrusion (or insertion portion) 12 which is inserted into a bone hole prepared in the femur for the AMB and at the other end with a handle 14 for grasping. A portion of the main body 11 close to the protrusion 12 forms a guide pin insertion portion 16, through which a guide pin insertion hole or passage 18 extends from top to bottom (in a direction of inserting the guide pin) in FIG. 1. The guide pin insertion hole 18 includes a guide pin guiding portion (or guide pin guiding surface) 18A, which is inclined at an angle α (FIG. 2) with respect to an axis of the main body in a horizontal plane so that the desired inserting direction can be obtained and which is inclined at an angle β in a perpendicular plane (FIG. 3). The guiding portion 18A has a substantially semi-circular cross section (a radial center line of this semi-circular guiding portion 18A is shown by the dashed line on the cross section in FIGS. 2 and 3) in a plane that intersects the guide pin inserting direction at right angles. The diameter of this semicircle corresponds to the diameter of the guide pin, so that the guide pin can be guided (or inserted) therein smoothly without a play along the guiding portion 18A.

The guide pin insertion hole 18 extends obliquely downwards with a width equal to the outer diameter of the guiding portion 18A and is open to a bottom surface of the main body, as shown in FIG. 1, so that the opening portion extending from the guiding portion 18A and having the same width forms a slot 20 of the present invention, which allows the guide pin guiding device 10 to be removed after the guide pin is inserted through the guide pin insertion hole 18 and pierced into the femur. The position and the direction of the guide-pin insertion hole 18 (particularly, the angles α and β) are determined as described below with reference to the AMB position.

In addition to the guide pin insertion hole 18, another set of a guide pin insertion hole and a slot can be provided in a portion at a distance away from it, so that a suitable one of these guide pin insertion holes can be used depending on a physical feature such as a body shape. Further, a disc-shaped angle gauge 22 (FIGS. 1 and 4) is disposed near the handle 14 so that it enables an operator to proceed with the operation while instantaneously recognizing the angle of the guide pin guiding device 10 with reference to the scales on the angle gauge 22.

The guide pin guiding device 10 is of two types, one for the left femur (shown in FIGS. 2A and 3A) and one for the right femur (shown in FIGS. 2B and 3B), and two types of the guide pin guiding device are different from each other in that the guide pin insertion holes 18 of the guide pin guiding devices for the left femur and for the right femur are located symmetrically.

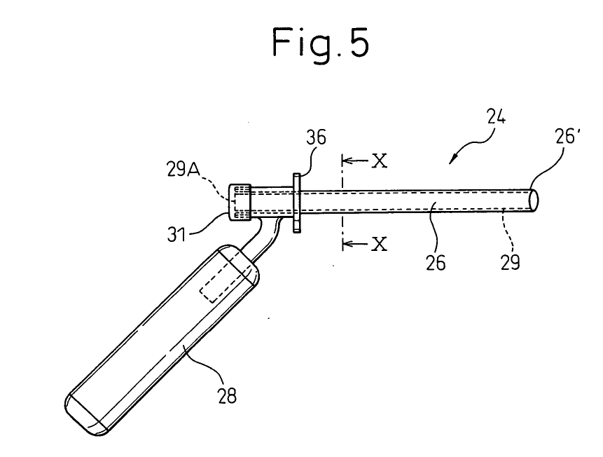

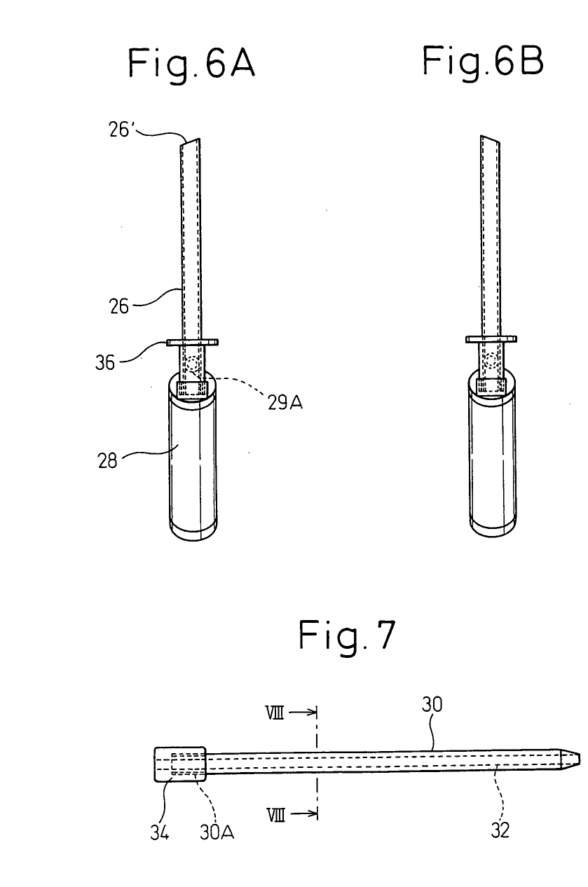

FIG. 5 shows a reamer guiding device 24 for guiding a reamer, which includes an outer tubular body 26 and a handle 28 located on one end of the outer tubular body 26. The outer tubular body 26 is formed with a reamer introduction hole 29 that extends through the outer tubular body 26 from one end to the other end thereof. A slightly beveled front end surface 26′ of the outer tubular body 26 (FIGS. 6A and 6B) is brought in contact with the intercondylar fossa wall, and the reamer is introduced into the reamer introduction hole 29 from the side of an external end 29A thereof, in this state, to prepare a bone hole in the femur.

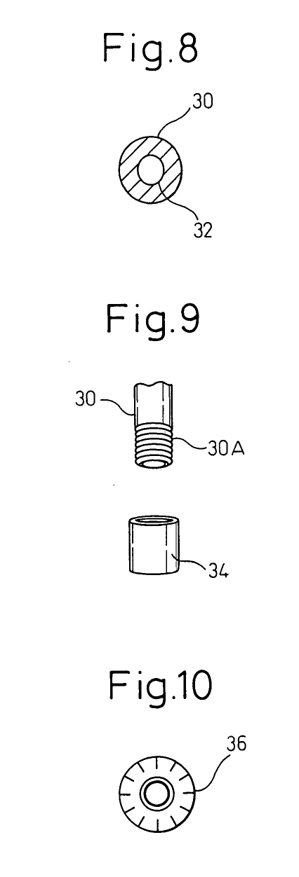

The reamer guiding device 24 is provided in two types for the left femur (FIG. 6A) and for the right femur (FIG. 6B), which are different from each other in that the angle of the front end surface 26′ is symmetrical for left and right femurs. The reamer guiding device 24 is intended to be used in conjunction with an inner tube 30 for preparing a bone hole, and the outside diameter of the inner tube 30 is adapted to be inserted without a play into the inside diameter of the reamer introduction hole 29 (FIG. 8) of the outer tubular body 26 of the reamer guiding device 24. A cover 31 may be provided for the outer tubular body 26.

On the other hand, the inner tube 30 is formed with a center hole 32 extending therethrough, and has a size which allows the insertion of the guide pin (K-wire) into the center hole 32 without play. As shown in FIG. 7, the outer end of the inner tube 30 is formed with a threaded portion 30A, onto which a cap 34 is threadedly mounted (see FIG. 9). The cap 34 is normally mounted onto the inner tube 30. However, when a hole is prepared, it is removed from the inner tube 30.

An angle gauge 36 (FIGS. 5 and 10) is disposed near the handle 28. Scales are marked on the angle gauge 36 so that the operator can read the indication of the scales thereby to instantaneously recognize the angle of the reamer guiding device 24.

Next, the procedure for preparing the bone hole according to the present invention, in which the anterior cruciate ligament is reconstructed by being divided into the AMB and the PLB, will be described. Please note that this procedure is arthroscopically carried out. Firstly, a knee of a patient is bent at 90° of flexion by an Alvarado fixator and held in a position where the knee cannot be rotated to the tibia. In preparing bone holes for the AMB in the tibia and the femur, a guide pin is pierced, for example, by the use of an ACL guide system available by Linvatec Corporation, U.S.A.

A target point is determined by connecting three points, an anteroposterior center of the tibia attachment sites (specifically, a peak of a nutrient vessel or an intercondylar eminence), a lateral edge of the posterior cruciate ligament (PCL), and a point from 3-6 mm ahead from a posteriormost edge (over the top) of the intercondylar fossa. Then, a C-reamer is used to prepare a hole having a diameter of 10 mm.

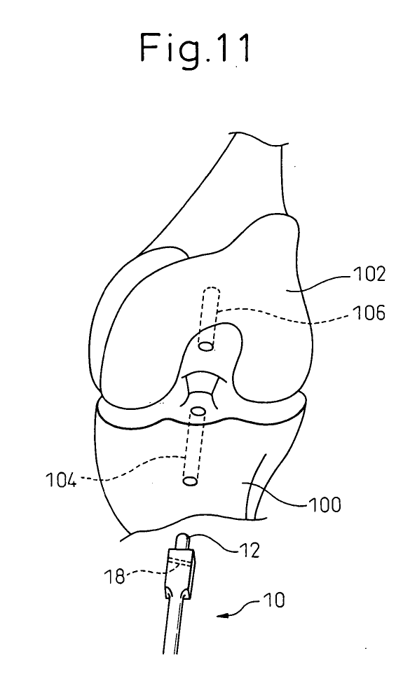

FIG. 11 schematically illustrates the left tibia 100 and the left femur 102 bent at 90° of flexion as viewed from the front, in which as described above, reference numeral “104” denotes the bone hole prepared in the tibia 100 of the front knee bent and reference numeral “106” denotes the bone hole prepared in the femur 102. The bone hole 104 of the tibia 100 is commonly used for the AMB and the PLB, while the bone hole 106 is used only for the AMB.

Next, a procedure of preparing a bone hole for the PLB in the femur will be described below.

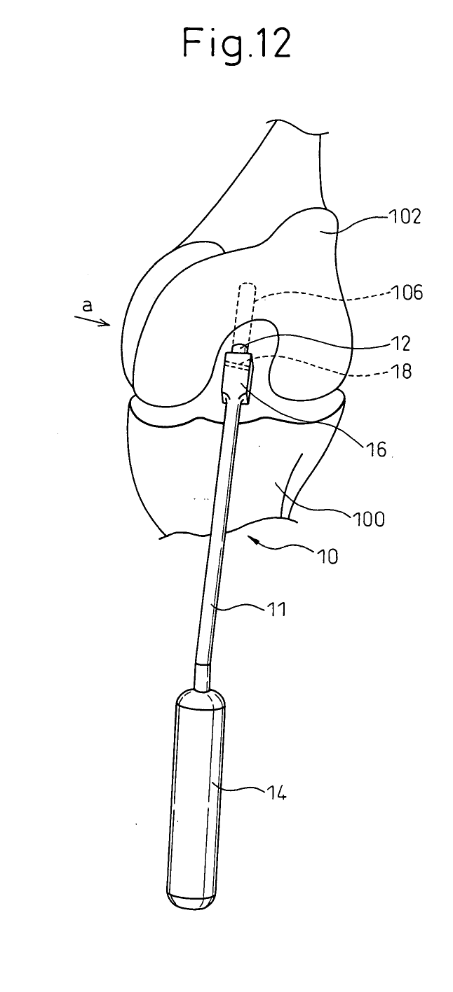

The guide pin guiding device 10 is positioned in the front of the knee joint, and the protrusion 12 is then inserted into the bone hole 104 for the AMB prepared in the tibia 100 as described above. FIG. 12 shows the protrusion inserted into the bone hole 104. In this state, the angle gauge 22 is used to correctly adjust the position of the guide pin guiding device 10. Then, the guide pin (2.4 mm in diameter) is pierced from the posteromedial side (in a direction indicated by an arrow “a”) into the guide pin insertion hole 18 of the guide pin insertion portion 16. The guide pin is inserted along the guiding surface 18A of the guide pin insertion hole 18 as if it is attached to the guide surface 18A. Therefore, by properly determining a curvature radius of the guiding surface 18A (having a semi-circular cross section) depending on the diameter of the guide pin, a smooth guidance without a play can be achieved. The guide pin is pierced in a direction toward a point positioned at 5-6 mm ahead of the femoral posterior condylar joint cartilage edge and on the lower edge of the bone hole 104 for the AMB (at an angle of 80-90° with reference to the PCL), and a guide pin insertion hole 18 is designed to obtain such a positional relationship.

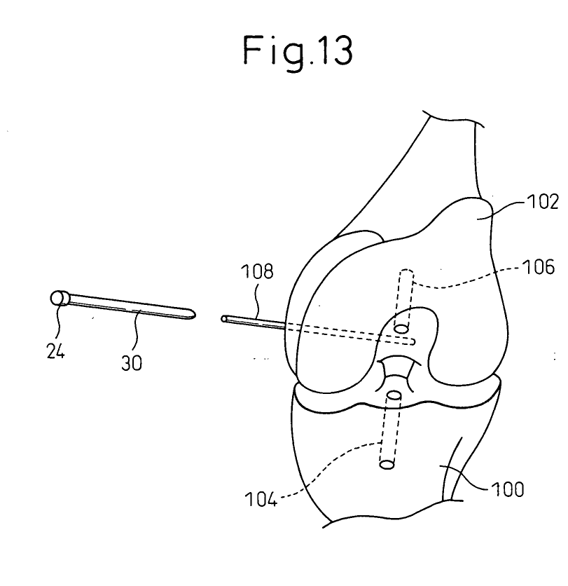

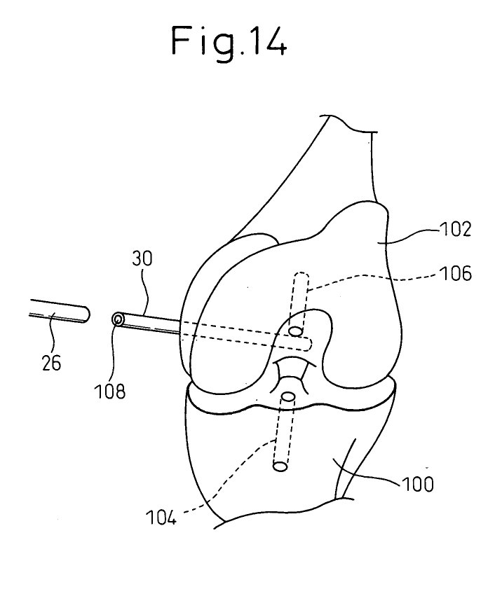

Such piercing of the guide pin from the posteromedial side is an operation required to be carried very carefully in order to protect the joint capsule tissue and further requiring high skill. However, in the present invention, as the guide pin insertion hole 18 is formed to extend through the guide pin guiding device 10 in the above-mentioned direction, the operation can be carried out properly, without requiring great skill with the aid of the guide pin insertion hole 18 (particularly, guiding surface 18A defined by the angles α and β) as long as the guide pin guiding device 10 is properly positioned. FIG. 13 illustrates a guide pin 108 pierced from the posteromedial side into the side wall of the intercondylar fossa of the femur. After the guide pin 108 is pierced into the intercondylar fossa, the guide pin guiding device 10 is removed while the guide pin 108 is still pierced therein. This operation can be carried out without any interference due to the presence of the slot 20 connecting to the guiding surface 18A and opening to the bottom surface.

Then, the inner tube 30 is inserted into the guide pin 108 with the cap 34 threadedly mounted on the end of the inner tube 30. Next, as shown in FIG. 14, the cap 34 at the end of the inner tube 30 is removed, and the outer tubular body 26 of the reamer guiding device 24 is inserted into the inner tube 30. Although there is a difference between the diameter of the guide pin and the diameter of the reamer, the use of the inner tube 30 having an intermediate diameter allows the reamer guide 24 to be properly positioned, at a portion of the femur, for the bone hole to be prepared.

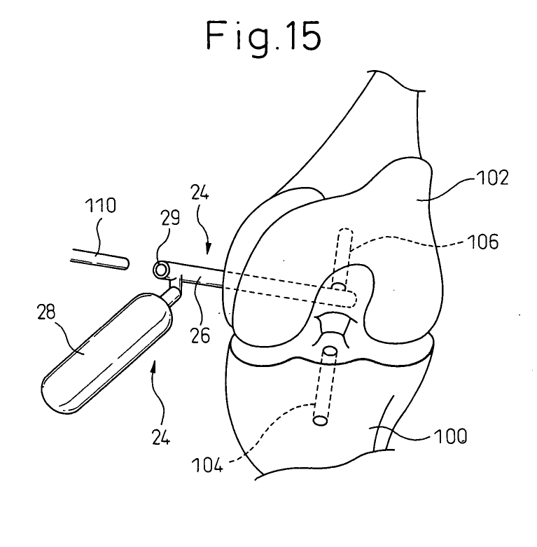

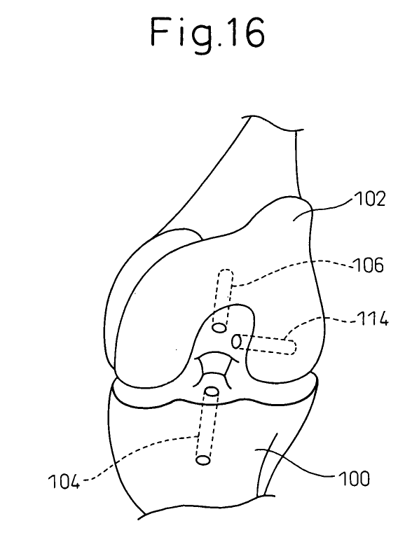

Then, the inner tube 30 and the guide pin 108 are pulled out while leaving the outer tubular body 26 of the reamer guide 24 engaged and retained to the wall of the intercondylar fossa of the femur at the beveled front end surface 26′ (see FIG. 6). FIG. 15 shows a state of the reamer guide 24 at this time, in which the reamer guide 24 is positioned at the “posteromedial side”. When the inner tube 30 and the guide pin 108 is pulled out as shown in FIG. 15, the cutting edge of the reamer 110 is introduced into the reamer introduction hole 29 of the outer tubular body 26 of the reamer guide 24 to prepare a bone hole on the side wall of the intercondylar fossa of the femur by the reamer 110. FIG. 16 shows a state in which the piercing operation (over-reaming) by the reamer 110 is completed thereby to prepare a bone hole 114 for the PLB in the femur.

In this way, after the completion of preparing the bone holes in the tibia and the femur, the anterior cruciate knee ligament (ACL) are functionally reconstructed. That is, in the embodiment of the present invention, a semitendinous muscle tendon (ST) is used for the reconstruction of the functions of the AMB and while a gracilis muscle tendon (Gr) is used for the reconstruction of the functions of the PLB. Specifically, the semitendinous muscle tendon (ST) for the AMB is used in a state of a fourfold loop, with one end inserted into the bone hole 104 of the tibia and the other end inserted into the bone hole 106 of the femur. On the other hand, the gracilis muscle tendon (Gr) for the PLB is used in a state of a double or triple loop, with one end inserted into the bone hole 104 of the tibia together with the AMB and the other end inserted into the bone hole 114 of the femur. In this way, two fiber bundles inserted in the joint are fixed as follows: firstly, the semitendinous muscle tendon (ST) for the AMB is fixed with the knee joint bent at 20° of flexion angle, and the gracilis muscle tendon (Gr) is then fixed with the knee joint bent at 90° of flexion angle. That is, these ST and Gr are fixed by means of a post screw in accordance with the usual procedure with the manual maximum tension applied thereto.

While the present invention has been described with reference to the embodiments shown in the accompanying drawings, these embodiments are only illustrative but not restrictive. Therefore, the scope of the present invention is limited by the appended claims and the preferred embodiments of the present invention can be modified or changed without departing from the scope of the claims.

1. A guide pin guiding device for guiding a guide pin when a bone hole for a posterolateral fiber bundle is prepared in the femur during a reconstruction procedure in which an anterior cruciate knee ligament is reconstructed by being divided into an anteromedial fiber bundle and the posterolateral fiber bundle, said guide pin guiding device comprising:
an elongate main body;
an insertion portion formed at an end of said elongate main body, said insertion portion inserted into a bone hole prepared in the femur for the anteromedial fiber bundle; and
a guide pin insertion hole formed in a portion of said elongate main body adjacent to said insertion portion for said guide pin to be inserted therein to prepare a bone hole for the posterolateral fiber bundle in the femur, said guide pin insertion hole being open to the outside of said elongate main body.

2. The guide pin guiding device according to claim 1, wherein said guide pin insertion hole comprises a guide pin guiding portion and a slot portion extending from said guide pin guiding portion to a side edge of said elongate main body.

3. The guide pin guiding device according to claim 1, wherein a plurality of guide pin insertion holes are formed in said elongate main body.

4. A reamer guiding device for guiding a reamer for preparing a bone hole for a posterolateral fiber bundle in a femur during a reconstruction procedure in which an anterior cruciate knee ligament is reconstructed by being divided into an anteromedial fiber bundle and the posterolateral fiber bundle, said reamer guiding device comprising:
an outer tubular body formed with a through-hole extending therethrough from one end to the other end thereof for a guide pin and an auxiliary inserting tool to be inserted therein, said guide pin pierced in the femur to prepare a bone hole for the posterolateral fiber bundle therein; and
a handle portion provided at one end of said outer tubular body.

5. A combination of a guide pin guiding device and a reamer guiding device. an elongate main body; an insertion portion formed at an end of said elongate main body, said insertion portion inserted into a bone hole prepared in the femur for the anteromedial fiber bundle; and a guide p)in insertion hole formed in a portion of said elongate main body adjacent to said insertion portion for said guide pin to be inserted therein to prepare a bone hole for the posterolateral fiber bundle in the femur, said guide pin insertion hole being open to the outside of said elongate main body; and an outer tubular body formed with a through-hole extending therethrough from one end to the other end thereof for a guide pin and an auxiliary inserting tool to be inserted therein, said guide pin pierced in the femur to prepare a bone hole for the posterolateral fiber bundle therein; and a handle portion provided at one end of said outer tubular body.
